(19) United States
(12) Patent Application Publication
(10) Pub. No.: US 2014/0371721 A1
(43) Pub. Date: Dec. 18, 2014

(54) SYSTEM AND METHOD FOR MIXING AND DISPENSING BIOMATERIALS

(71) Applicant: Nordson Corporation, Westlake, OH (US)

(72) Inventors: Benjamin B. Anderson, Wheeling, IL (US); Mark Stevenson, Cottage Grove, MN (US)

(21) Appl. No.: 14/306,748

(22) Filed: Jun. 17, 2014

Related U.S. Application Data
(60) Provisional application No. 61/835,780, filed on Jun. 17, 2013.

(51) Int. Cl. A61B 17/88

(52) U.S. Cl. CPC A61B 17/8825; A61B 17/8833; A61B 17/8822; A61B 2017/8838
USPC 604/522; 604/82

(57) ABSTRACT

A system for mixing and dispensing at least first and second components of a biomaterial. The system includes a first syringe including a barrel having a proximal and distal ends. The barrel is configured to receive the first and second components of the biomaterial. There is a cap on the distal end of the barrel. The cap has an opening to allow materials to be directed into the barrel. The system further includes a mixing member. At least a portion of the mixing member is configured to enter the barrel through the cap. The mixing member is configured to move at least axially within the barrel to mix the first and second components.

### CROSS-REFERENCE TO RELATED APPLICATION

This application claims the benefit of Application Ser. No. 61/835,780 filed Jun. 17, 2014 (pending), the disclosure of which is hereby incorporated by reference herein.

### FIELD OF THE INVENTION

The present invention generally relates to dispensing equipment, and more particularly to systems and methods used for mixing and dispensing biomaterials such as bone graft materials.

### BACKGROUND

Biomaterials have various uses in medical applications. For example, bone grafting is a surgical procedure for repairing bones and typically involves introducing a bone graft material (which is a type of biomaterial) into an area of bone that requires repair, such as a fracture. The bone graft material is intended to stimulate growth of healthy native bone tissue, and new native bone tissue may eventually replace the bone graft material completely. Bone graft material typically includes a combination of crushed bone and a liquid component, such as blood, plasma, or growth factors. Bone graft materials can be allograft (derived from a human other than the one receiving the graft), autograft (derived from the human receiving the graft), and synthetic (created from, for example, ceramics such as calcium phosphates).

The bone graft material is often applied to a treatment site using a syringe with a dispensing apparatus at the distal end, depending on the particular treatment site. The bone graft material utilized in such procedures often has two components—the first being a solid, such as powderized, granulated, or crushed bone, and the second being a liquid. The two components typically are mixed just prior to delivery to a treatment site. Therefore, the practitioner must add an amount of each of the first and second components into a mixing receptacle, mix the components, and pour the mixture into a dispensing syringe. This existing method of mixing often leads to waste of the expensive bone graft material, as the practitioner may mix more than is needed for the procedure. Moreover, waste may occur due to spillage as the bone graft material is transferred from the mixing receptacle to the delivery device, such as a syringe.

There are devices in the art which attempt to alleviate the problems described above. For example, one such device includes a syringe with a removable cap having an attached mixing rod. After multiple materials are directed into the syringe, such as by pouring, the cap is placed on the dispensing end of the syringe. The mixing rod is movable relative to the cap in order to mix the contents. Once the contents are mixed, the cap is then removed and the mixed materials may be dispensed. At least one issue that still remains as a challenge is that of loading one or more components into the syringe. There remains a need to address these and other challenges in the art.

### SUMMARY

To that end, a system for mixing and dispensing at least first and second components of a biomaterial is provided. The system includes a first syringe including a barrel having a proximal and distal ends. The barrel is configured to receive the first and second components of the biomaterial. There is a cap on the distal end of the barrel. The cap has an opening to allow materials to be directed into the barrel. The system further includes a mixing member. At least a portion of the mixing member is configured to enter the barrel through the cap. The mixing member is configured to move within the barrel to mix the first and second components. The movement, for example, may be rotational and/or axial along the barrel. In one embodiment, the system further includes a supply syringe configured to be fluidicly communicated with the opening for injecting at least one of first or second components of the biomaterial into the barrel.

The invention further includes a method of mixing and dispensing at least first and second components of a biomaterial. The method includes filling at least part of a first syringe with at least one of the first or second components. A cap is attached to a distal end of the first syringe, and the cap includes an opening. The other of the first or second components is directed into the first syringe through the opening in the cap. The mixing member in the first syringe is then moved to mix the components, with at least a portion of the mixing member entering the barrel through the cap.

Various additional aspects and features of the invention will become more readily apparent to those of ordinary skill in the art upon review of the following detailed description of the illustrative embodiments.

### BRIEF DESCRIPTION OF THE DRAWINGS

FIG. 1 is a perspective view of a system for mixing and dispensing multiple components of a biomaterial according to one embodiment.

FIG. 2 is an exploded perspective view of various components of the system shown in FIG. 1.

FIG. 3A is a side cross-sectional view of the first syringe of the system of FIG. 1, generally taken along line 3A-3A of FIG. 1, but showing the mixing member retracted into the first syringe.

FIG. 3B is a side cross-sectional view similar to FIG. 3A, but shown with a supply syringe attached.

FIG. 3C is a side cross-sectional view similar to FIG. 3B but showing the supply syringe detached, and the first and second components of the biomaterial being mixed.

FIG. 4 is a perspective view of the system shown in FIG. 3B.

FIGS. 5A and 5B are perspective and top plan views, respectively, of one embodiment of a mixer.

FIGS. 6A and 6B are perspective and top plan views, respectively, of an alternative embodiment of a mixer.

FIGS. 7A and 7B are perspective and top plan views, respectively, of yet another alternative embodiment of a mixer.

FIGS. 8A and 8B are perspective and top plan views, respectively, of yet another alternative embodiment of a mixer.

FIGS. 9A and 9B are perspective and top plan views, respectively, of yet another alternative embodiment of a mixer.

### DETAILED DESCRIPTION

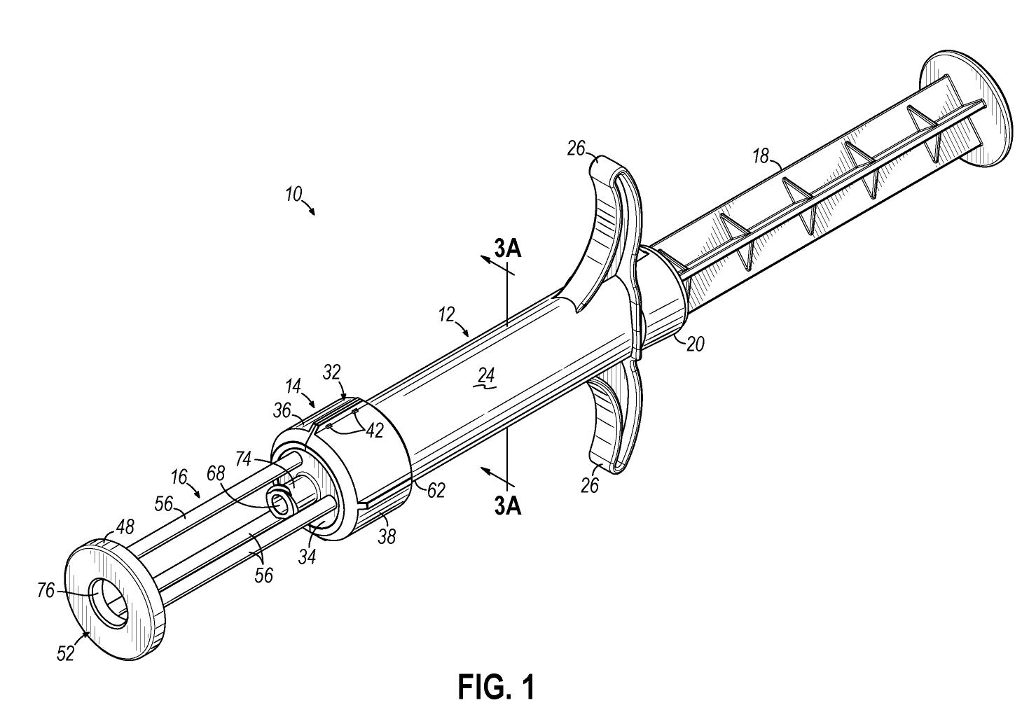

Referring to the figures, and beginning with FIGS. 1-4, an exemplary system 10 is shown and includes a first syringe 12, a cap 14, and a mixing member 16. The system 10 also includes a plunger 18, which seals a proximal end 20 of the first syringe 12. As will become apparent from the following description, the system 10 is used to mix a biomaterial comprised of multiple components. For example, in the embodiments shown and described, the biomaterial that is mixed and eventually dispensed is a bone graft material, comprising a first, solid component, and a second, liquid component such as described above. Once mixed, a dispensing cap or apparatus (not shown) may be attached to the distal end 22 of the first syringe 12 in order to deliver the biomaterial to a treatment site.

The first syringe 12 further includes a barrel 24 between the proximal and distal ends 20, 22. The barrel 24 may include finger grips 26 extending radially therefrom in order to assist the user in depressing the plunger 18. However, the finger grips 26 may alternatively comprise a radially extending flange or another configuration that assists the user in depressing the plunger 18. The plunger 18 is inserted into an opening 28 at the proximal end 20 of the first syringe 12 and movable within the barrel 24 in order to dispense materials from the distal end 22. In order to counteract the changes in pressure and allow the release of air during mixing, the barrel 24 and/or the plunger 18 may be configured to allow the escape of air out of the opening 28 in the first syringe 12. In one embodiment, the plunger 18 includes a gas permeable membrane 30 in order to allow the escape of gas out of the opening 28. The membrane 30, however, is preferably liquid impermeable, as described below.

The cap 14 includes a housing 32 and a lid 34. The housing 32 includes first and second portions 36, 38 that are able to be easily assembled and disassembled. In order to assemble the first and second portions 36, 38, one or both of the first and second portions 36, 38 includes features which allow attachment to one another. In the embodiment shown, the first portion 36 includes pegs 40 which are received into corresponding apertures 42 of the second portion 38. In one embodiment, the pegs 40 may be directed into the apertures 42 and resiliently engaged therein. The attachment features which allow attachment between the first and second portions 36, 38 are, however, not limited to those shown in the figures.

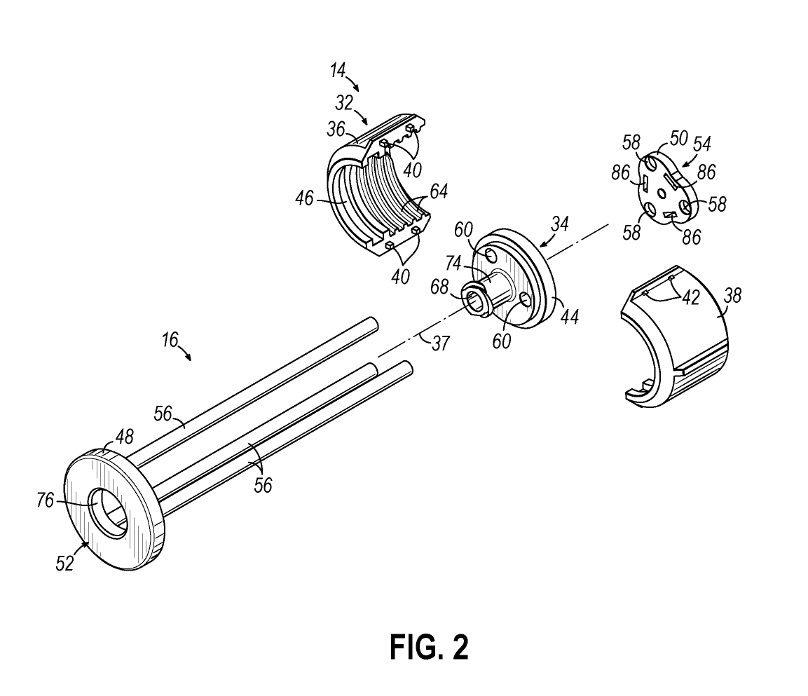

The first and second portions 36, 38 of the housing 32 are attached and assembled together, along with the lid 34, such that the cap 14 is thereby assembled. More specifically, the first and second portions 36, 38 of the housing 32 are attached together and relative to the lid 34 such that the annular rib 44 of the lid 34 is received into the annular recess 46 of the housing 32 as will be apparent from FIGS. 2 & 3A through 3C.

The mixing member 16 includes a proximal end 48 and a distal end 50. At the proximal end 48, there is a gripping member 52, which provides an area for a user to grip during use of the system 10. At the distal end 50, there is a mixer 54 which, as discussed hereinbelow, includes features that are provided for allowing a user to thoroughly mix the components of the biomaterial. Between the proximal and distal ends 48, 50, the mixing member 16 includes a plurality of elongate members 56. The elongate members 56 provide connection between the gripping member 52 and the mixer 54. The mixer 54 includes a plurality of apertures 58 corresponding to the shape and the number of elongate members 56 in order to fix the mixer 54 relative to the elongate members 56 and the gripping member 52. The gripping member 52 also may include a plurality of bores or apertures (not shown) which receive the elongate members 56 for connection therebetween. Alternatively, however, the elongate members 56 may be manufactured as integral members with the mixer 54 or the gripping member 52. As shown, there are three elongate members 56. In alternative embodiments, there may be more or less elongate members 56. The elongate members 56 may be of a different shape than the cylindrical shape shown. In addition to providing connection between the mixer 54 and the gripping member 52, the elongate members 56 also play a role in mixing the biomaterial within the barrel 24. More specifically, the elongate members 56 mix the biomaterial as the mixing member 16 is rotated. The elongate members 56 may mix the biomaterial as the mixing member 16 is moved axially, depending on the amount of friction between the elongate members 56 and the biomaterial and/or the use of any mixing elements (not shown) along the elongate members 56.

The lid 34 includes a plurality of apertures 60 corresponding to the size, location and shape of the elongate members 56. The elongate members 56 may be directed through the apertures 60 and move axially within and relative to the apertures. The axial movement of the elongate members 56 provides axial movement of the mixer 54, for mixing of the biomaterial within the barrel 24. Because the lid 34 is rotatable relative to the housing 32, the user may also rotate the mixing member 16 relative to a central axis 37 of the housing 32 to aid in the mixing process. As discussed below, the mixer 54 may include features which are configured to further mix the biomaterial as the mixing member 16 is rotated.

Once assembled, the cap 14, with the mixing member 16, may be attached to the distal end of the barrel 24. In order to facilitate attachment between the cap 14 and the distal end 62 of the barrel 24, there may be an attachment mechanism on one or both of the cap 14 and the barrel 24. As shown, the cap 14 and the barrel 24 each include corresponding threaded portions 64, 66, which allow for threaded engagement therebetween. The cap 14 may be screwed onto the barrel 24 such that the threaded portions 64, 66 are engaged, thereby fixing the cap 14 relative to the barrel 24.

It is desirable to prevent the escape or leakage of the biomaterial from the barrel 24 at several areas of the system 10. In order to prevent such leakage at various vulnerable areas of the system 10, there may be features provided on various parts of the system 10 which are configured to prevent the leakage or escape of biomaterial from the barrel 24. For example, the plunger 18 may include the gas-permeable, liquid impermeable membrane 30 such that gas is allowed to escape from the distal end 22 of the first syringe 12, while liquid cannot. There may be features in between the lid 34 and the housing 32 that would prevent leakage at the junction thereof, such as an O-ring (not shown) or other sealing element(s). The same may be provided at the apertures 60 so that biomaterial does not leak from between the aperture 60 and the elongate members 56.

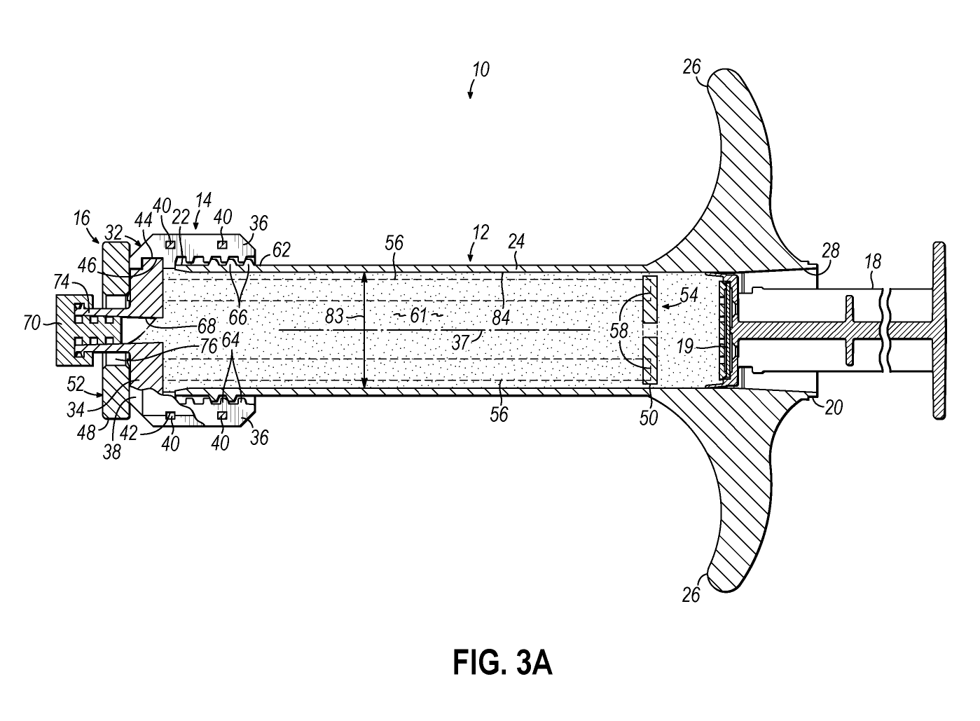

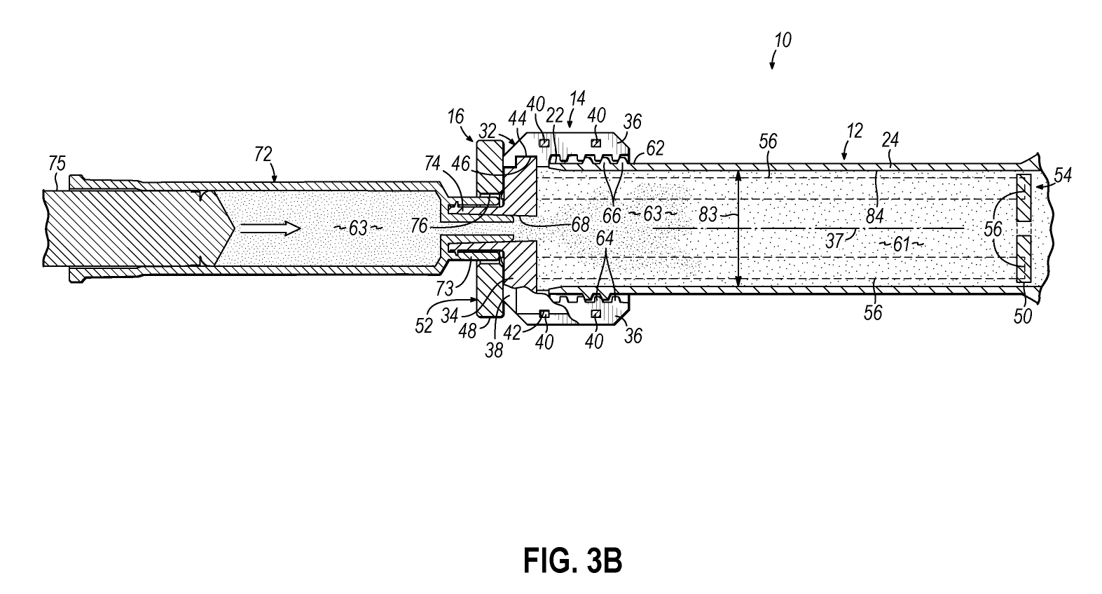

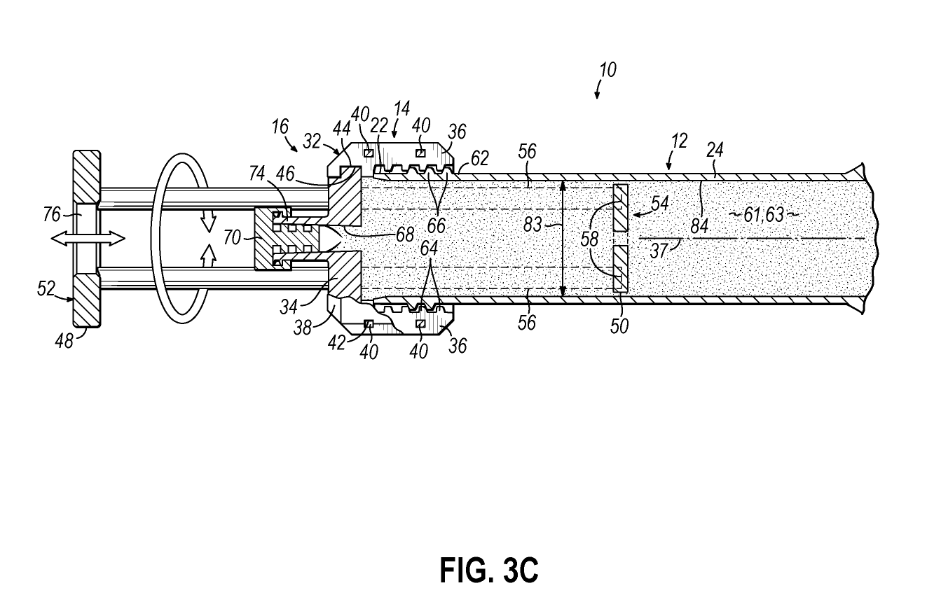

With reference to FIG. 3A, the first syringe 12 has the first, solid component 61 of the biomaterial loaded therein. As shown, the first component 61 is pre-packaged into the first syringe outside of the operating theater. As described herein, pre-packaged means that the components are added to their respective syringes in a location such as a packaging or manufacturing facility, before entering the operating theatre. In order to prevent unwanted escape of the first component 61 from the opening 68, a plug 70 is provided in the opening. As shown, the mixing member 16 is moved distally such that the gripping member 52 is substantially in contact with the cap 14.

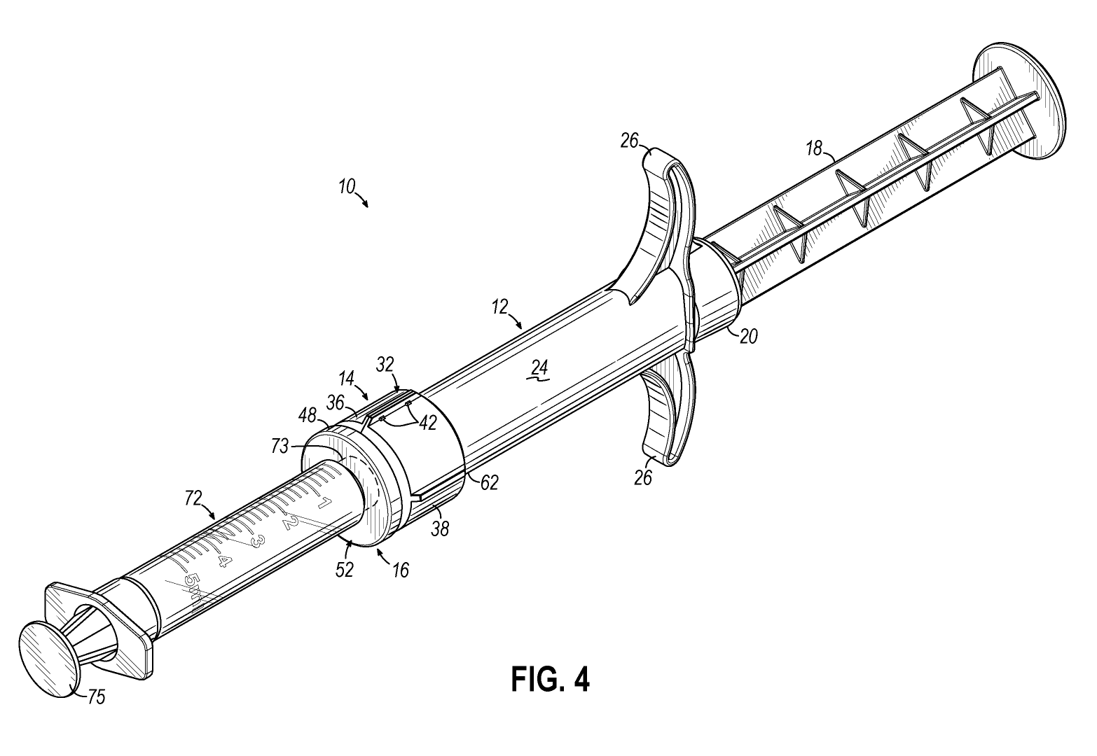

As shown in FIGS. 3B, 3C, and 4, the system 10 includes a supply syringe 72. The supply syringe 72 is fluidicly communicated with the opening 68 in order to allow the user to inject the second component 63 of the biomaterial into the barrel 24. More specifically, the plug 70 is removed from the opening 68. Once the plug 70 is removed, the practitioner may then couple or engage a luer connector 73 of the supply syringe 72 with the corresponding luer connector 74 of the cap 14 such that the first syringe 12 and supply syringe 72 are in fluid communication.

Once the first syringe 12 and supply syringe 72 are in fluid communication, the user may depress the plunger 75 of the supply syringe 72 to inject the second, liquid component 63 of the biomaterial into the barrel 24. After injecting the second component 63 from the supply syringe 72 into the first syringe 12, the user may then mix the first and second components 61, 63 in the barrel 24 using the mixing member 16. Mixing is accomplished by rotational and axial movement of the mixing member 16, as represented by arrows in FIG. 3C. As best seen in FIG. 3B, the outer diameter of the supply syringe 72 is greater than the diameter of the aperture 76 in the gripping member 52. As such, the mixing member 16 is unable to be moved axially with respect to the rest of the system 10 in order to mix the contents of the barrel 24. However, with other sizes of supply syringes 72, where the effective diameter thereof is smaller than the diameter of the opening 76, the mixing member 16 is able to move axially with respect to the system 10 while the supply syringe is still attached.

However, in the event that the effective diameter of the supply syringe 72 is larger than that of the opening 76, the practitioner would be required to decouple the luer connectors 73, 74 in order remove the supply syringe 72 from the system 10. The practitioner would then replace the plug 70 to essentially seal the opening 68. Once the plug 70 is replaced in the opening 68, the practitioner would be able to mix the contents of the barrel 24 without contents spilling from the opening 68. Then, in order to mix the contents, the mixing member 16 is moved axially and/or rotationally, as shown by the arrows in FIG. 3C. The mixer 54 (described below) and elongate members 56 (described above) each play a role in the mixing process. Different embodiments of the mixer 54 are described below and include various features to facilitate mixing of the contents of the barrel 24.

In one embodiment, the first syringe 12 and the supply syringe 72 are pre-packaged with a supply of the first and second components 61, 63, respectively. Alternatively, one or both of the first syringe 12 and supply syringe 72 may be loaded with their respective components of the biomaterial in the operating or pre-operating settings, or at a time deemed appropriate by the practitioner, his staff, or other individuals involved. Notably, where the first component 61 is the solid component (i.e., crushed bone) of the biomaterial, the second component 63 is the liquid component of the biomaterial, and vice versa. As described, the first syringe 12 contains the solid component 61 of the biomaterial, and the supply syringe 72 contains the liquid component 63, until the liquid component 63 is directed into the barrel 24 of the first syringe 12. Alternatively, however, the first syringe 12 may contain the liquid component 63 and the supply syringe 72 may contain the solid component 61. Once sufficiently mixed, the user may remove the cap 14 and mixing member 16 and fix an attachment (not shown) suitable for dispensing the biomaterial from the first syringe 12.

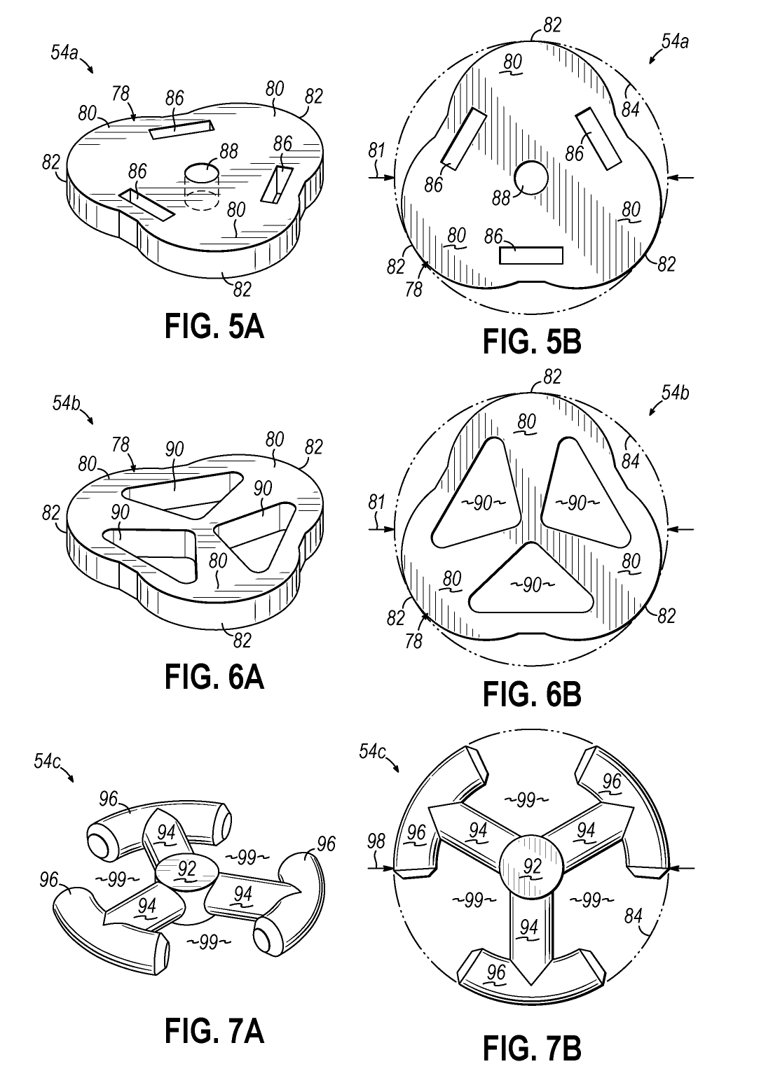

FIGS. 5A-5B, 6A-6B, 7A-7B, 8A-8B, and 9A-9B each show alternative embodiments of mixers 54a, 54b, 54c, 54d, and 54e, respectively. With reference to FIGS. 5A-5B and 6A-6B, the mixer 54a includes a body 78 having a lobed configuration. More specifically, the body 78 of the mixer 54a includes three lobes 80. The body 78 is configured such that the effective diameter 81 of the mixer 54 is substantially equal to or slightly less than the inner diameter 83 (FIG. 3) of the barrel 24. In other words, the mixer 54a is configured such that the outer most portions 82 of the lobes 80 contact the inner wall 84 of the barrel 24. This enables the mixer 54a to scrape the contents of the barrel 24 from the inner wall 84, thus enabling a more effective mixing procedure. The lobed configuration also allows material to travel around the mixer 54a, thereby further enabling mixing, as well as preventing the buildup of pressure as the mixing member 16, and thus the mixer 54a, is moved rotationally and axially within the barrel 24. The mixer 54a further includes a plurality of apertures 86, which also aid in the mixing process and prevent the buildup of pressure within the barrel 24 by allowing material to pass therethrough. As shown in FIGS. 5A and 5B, the apertures 86 are rectangular. The disclosure herein, however, is not meant to be limited to these shapes. For example, the apertures 86 may be circular, elliptical, various quadrilateral shapes, and so on. The mixer 54a includes bore 88, which may be provided for similar reasons as apertures 86, or for providing connection to one of the elongate members 56 (provided that at least one elongate member 56 is in a corresponding position).

FIGS. 6A and 6B show a mixer 54b, which is an alternative embodiment of the mixer 54a. Many of the components of this embodiment of the mixer 54b are identical or substantially similar to the components described above with reference to the embodiment shown in FIGS. 5A and 5B, and these components have been marked with the same reference numbers in this embodiment without additional explanation below. To this end, mixer 54b includes alternative apertures 90, shown as essentially triangular shapes with rounded corners. Apertures 90 aid in the mixing process and prevent the buildup of pressure within the barrel 24 by allowing material to pass therethrough during the axial movement of the mixing member 16.

As shown in FIGS. 7A and 7B, the mixer 54c includes a center member 92 a plurality of radially extending appendages 94. Each of the appendages 94 includes an arm 96 situated essentially transversely to each respective appendage 94. The effective diameter 98 of the mixer 54c is substantially equal to or slightly less than the inner diameter of the barrel 24. In other words, the mixer 54c is configured such that the outer most portions of the arms contact the inner wall 84 of the barrel 24. In other words, the mixer 54c is configured such that the arms 96 contact the inner wall 84 of the barrel 24. This enables the arms 96, which may have an essentially matching curvature with the inner wall 84 of the barrel 24, to scrape the contents of the barrel 24 from the inner wall 84, thus enabling a more effective mixing procedure. For further facilitation of mixing, there is a plurality of spaces 99, each space 99 defined as a void between two appendages 94. As the mixing member 16 is moved axially and rotationally, the contents of the barrel 24, such as the first and second components of the biomaterial, are mixed to be suitable for delivery to a treatment site.

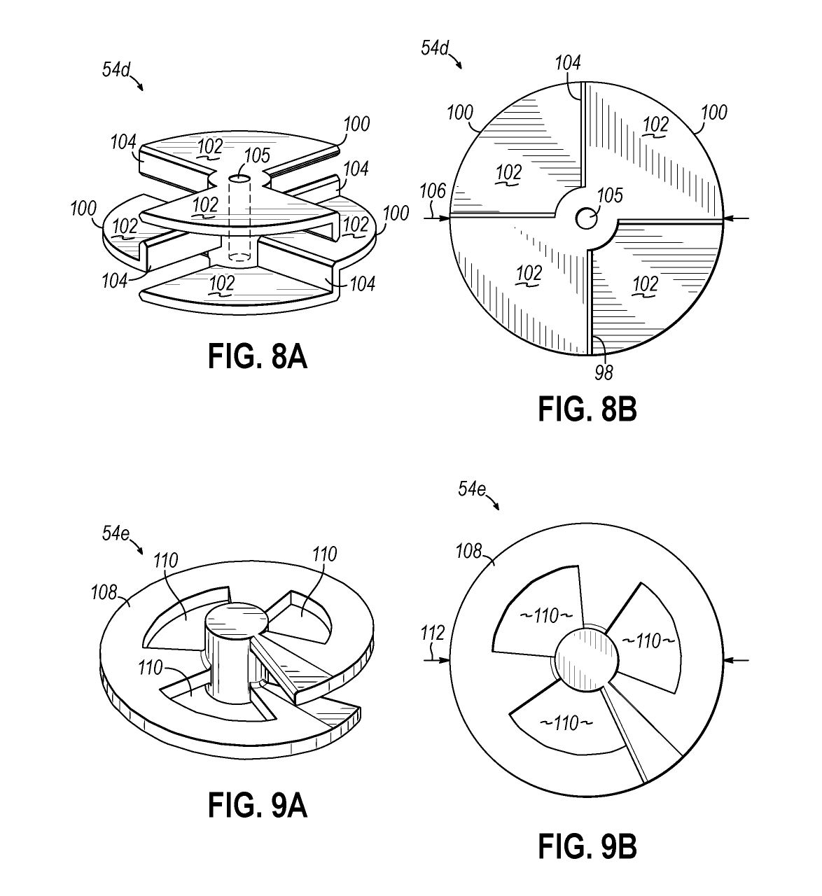

Yet another alternative embodiment of a mixer 54d is shown in FIGS. 8A and 8B. As shown, mixer 54d includes blades 100 shaped as variously sized sector-shaped portions 102. More specifically, sector-shaped portions 102 are shaped as sectors of a circle. The blades 100 further include transverse portions 104, which are situated transversely to the sector-shaped portions 102. Similar to the other embodiments, the effective diameter 106 of the mixer 54d is substantially equal to or slightly less than the inner diameter of the barrel 24 so that at least some portions of the mixer 54d may scrape the contents of the barrel 24 from the inner wall 84. The bore 105 may be provided in order to couple the mixer 54d with an elongate member 56.

Another alternative embodiment of a mixer 54e is shown in FIGS. 9A and 9B. As shown the mixer 54e is defined in part a single, helical blade 108 having apertures 110 similar to those shown in FIGS. 6A and 6B. As with the other embodiments, the effective diameter 112 of the mixer 54e is substantially equal to or slightly less than the inner diameter of the barrel 24 so that at least some portions of the mixer 54e may scrape the contents of the barrel 24 from the inner wall 84. The configurations as shown in FIGS. 8A, 8B, 9A, and 9B, allow material to travel around the mixer 54d, 54e, respectively, thereby further enabling mixing. Such configurations also serve the purpose of preventing the buildup of pressure as the mixing member 16, and thus the mixers 54d, 54e, are moved rotationally and axially within the barrel 24.

The mixers 54a, 54b, 54c, 54d, and 54e may be coupled to or connected with the elongate members 56 in a variety of manners. As discussed above, the mixers 54a, 54b, 54c, 54d, and 54e may be manufactured as an integral component with the elongate members 56, such as by injection molding. Alternatively, the mixers 54a, 54b, 54c, 54d, and 54e may have one or more bores (such as bores 88, 105 in FIGS. 5A and 5B, and 8A and 8B respectively), into which the elongate members 56 are inserted to thereby fix the mixers 54a, 54b, 54c, 54d, and 54e to the elongate members 56. Other methods of manufacturing different components of the system 10 will be apparent to persons skilled in the art.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A system for mixing and dispensing at least first and second components of a biomaterial, comprising:
a first syringe including a barrel having a proximal and distal ends, the barrel configured to receive the first and second components of the biomaterial;
a cap on the distal end of the barrel, the cap having an opening to allow materials to be directed into the barrel; and
a mixing member, at least a portion of the mixing member configured to enter the barrel through the cap, the mixing member configured to move within the barrel to mix the first and second components.

2. The system of claim 1, further comprising:
a supply syringe configured to be fluidicly communicated with the opening for injecting at least one of first or second components of the biomaterial into the barrel.

3. The system of claim 2, wherein the opening in the cap is defined in part by a luer connection to facilitate the fluidic communication between the supply syringe and the opening.

4. The system of claim 1, wherein the cap is removably attachable to the barrel, at least one of the cap or the barrel including an attachment structure for connection of the cap to the distal end of the barrel.

5. The system of claim 4, wherein the attachment mechanism is defined by a threaded portion on each of the cap and the barrel for threadable engagement therebetween.

6. The system of claim 1, wherein the cap further comprises:
a lid including the opening; and
a housing configured to secure the lid member relative to the barrel.

7. The system of claim 6, wherein the housing is configured to disassemble into first and second portions.

8. The system of claim 6, wherein the lid member is rotatable relative to the housing to enable rotation of the mixing member.

9. The system of claim 1, wherein the mixing member further comprises:
a proximal end;
a distal end including a mixer; and
at least one elongate member between the proximal and distal ends.

10. The system of claim 9, wherein the elongate members are configured to be accepted into and move axially within a plurality of apertures in the cap.

11. The system of claim 9, wherein the proximal end of the mixing member includes a gripping member for assisting a user in movement of the mixing member.

12. The system of claim 11, wherein the gripping member includes an aperture having a diameter larger than an effective diameter of the supply syringe.

13. The system of claim 1, further comprising:
a plunger inserted into the proximal end of the barrel.

14. The system of claim 13, wherein the plunger is configured to allow the escape of air from the proximal end of the barrel.

15. The system of claim 14, wherein at least of the portion of the plunger is defined by a gas-permeable material.

16. The system of claim 1, wherein:
the barrel includes a first component of the biomaterial; and
the supply syringe includes a second component of the biomaterial.

17. The system of claim 1, wherein the axial movement of the mixing member is limited in at least one of the proximal and distal directions by the cap.

18. A method of mixing and dispensing at least first and second components of a biomaterial, comprising:
filling at least part of a first syringe with at least one of the first or second components;
attaching a cap to a distal end of the first syringe, the cap including an opening;
directing the other of the first or second components into the first syringe through the opening in the cap;
moving a mixing member in the first syringe to mix the components, at least a portion of the mixing member entering the barrel through the cap.

19. The method of claim 18, further comprising:
fluidicly communicating a supply syringe with the opening;
injecting at least one of first or second components of the biomaterial into the barrel from the supply syringe.

20. The method of claim 18, further comprising:
rotating the mixing member and/or moving the mixing member axially along the barrel.
